(19) United States
(12) Patent Application Publication
(10) Pub. No.: US 2023/0158492 A1
(43) Pub. Date: May 25, 2023

(54) SET OF CHAMBERS CONTAINING REAGENTS

(71) Applicant: MIDGE MEDICAL GMBH, Berlin (DE)

(72) Inventors: Markus RIESTER, Wiesbaden (DE); Michael DIEBOLD, Berlin (DE)

(21) Appl. No.: 17/995,735

(22) PCT Filed: Apr. 7, 2021
(86) PCT No.: PCT/EP2021/059106

§ 371 Date: Oct. 7, 2022

(30) Foreign Application Priority Data
Apr. 7, 2020 (DE) 10 2020 109 744.1
Jun. 4, 2020 (DE) 10 2020 114 919.0
Aug. 21, 2020 (DE) 10 2020 121 996.2
Nov. 9, 2020 (EP) 20206554.6

(51) Int. Cl. B01L 3/00

(52) U.S. Cl. CPC B01L 3/502715; B01L 2200/16; B01L 2200/026; B01L 2300/087; B01L 2400/0683; B01L 2400/0478; B01L 2300/0832; B01L 2300/0672; B01L 2300/044

(57) ABSTRACT

The invention relates to a system is provided that comprises a first container, a second container and a fluorescence detection device. The first container comprises a first set of chemicals and/or agents and is closed prior to use. The second container comprises a second set of chemicals and/or agents that are at least in part distinct from the chemicals and/or agents of the first set. The first container comprises a lid, that can be opened when the first container and the second container are combined to form a single, fluid tight assembly, in order to allow the contents of the first container to enter the second container.

The invention relates to a set of chambers containing reagents to be used in a system and a device for detecting a target analyte, in particular a target nucleic acid, for instance DNA or RNA, by way of isothermal nucleic acid amplification and fluorescence.

Nucleic acid amplification technologies are used to amplify the amount of a target nucleic acid in a sample in order detect such target nucleic acid in the sample. A known nucleic acid amplification technology is Polymerase Chain Reaction (PCR). Isothermal nucleic acid amplification technologies offer advantages over polymerase chain reaction (PCR) in that they do not require thermal cycling or sophisticated laboratory equipment.

Known isothermal nucleic acid amplification technologies are inter alia Recombinase Polymerase Amplification (RPA) and Strand Invasion Based Amplification (SIBA) and other methods know to persons skilled in the art.

Recombinase polymerase amplification (RPA), is a method to amplify the amount of a target analyte, in particular a nucleic acid such as DNA or RNA in a sample. For recombinase polymerase amplification three core enzymes are used: a recombinase, a single-stranded DNA-binding protein (SSB) and a strand-displacing polymerase. Recombinases can pair oligonucleotide primers with homologous sequences in duplex DNA. SSB binds to displaced strands of DNA and prevents the primers from being displaced. The strand-displacing polymerase begins DNA synthesis at sites where the primer has bound to the target DNA. Thus, if a target gene sequence is indeed present in the tested sample, an exponential DNA amplification reaction can be achieved to amplify a small amount of a target nucleic acid to detectable levels within minutes at temperatures between 37° C. and 42° C.

The three core RPA enzymes can be supplemented by further enzymes to provide extra functionality. Addition of exonuclease III allows the use of an exo probe for real-time, fluorescence detection. If a reverse transcriptase that works at 37 to 42° C. is added then RNA can be reverse transcribed and the cDNA produced amplified all in one step.

By adding a reverse transcriptase enzyme to an RPA reaction, it can detect RNA as well as DNA, without the need for a separate step to produce cDNA. It is an advantage of RPA that it is isothermal and thus only requires simple equipment. While RPA operates best at temperatures of 37-42° C. it still works at room temperature.

For detecting the presence of a targeted nucleic acid in a sample, fluorescence detection technique can be used. After the light source at specific wavelength illuminates on the targeted nucleic acids, the DNA-binding dyes or fluorescein-binding probes of the nucleic acids will react and enable fluorescent signals to be emitted. The fluorescent signal is an indication of the existence of the targeted nucleic acids.

It is an object of the invention facilitate the testing of samples by means of nucleic acid amplification technology.

According to the invention, a set of containers is provided that comprises a first container, in particular a lysis container that encloses at least a first chamber and a second container, in particular a test container that encloses at least a second chamber.

The lysis container may contain a liquid lysing fluid that causes lysing of the cells in a sample to thus release the nucleic acids (DNA or RNA). The lysing fluid may comprise an acid, e.g. HCl or a weak alkali, and a surface active agent.

The test container contains a mixture of chemicals that can cause an amplification of nucleic acid in a sample. Preferably, the mixture comprises a recombinase, a single-stranded DNA-binding protein (SSB) and strand-displacing polymerase that causes a recombinase polymerase amplification (RPA). The test container further preferably contains exonuclease III allows the use of an exo probe for real-time, fluorescence detection. The mixture may be provided in form of a dry pellet.

According to a first aspect of the invention the first container, the second container and the fluid transfer interface are configured to allow a limited amount of fluid being transferred from the first chamber into the second chamber. The fluid transfer interface initially is closed so that the first container can be handled as a separate, closed unit.

In particular, a set of at least two distinct containers is provided that can be combined to form a single, fluid tight assembly, wherein a first container encloses at least a first chamber containing a first set of chemicals and/or agents and wherein a second container comprises at least a second chamber containing a second set of chemicals and/or agents that are at least in part distinct from the chemicals and/or agents of the first set of chemicals and/or agents. The first container and/or the second container comprises a septum that initially is fluid tight and that can be altered to form a passageway between the first container and the second container when the first container and the second container are combined to form a single, fluid tight assembly, in order to allow a controlled transfer of fluid from the first container to the second container.

Preferably, the first container and the second container are arranged in a housing that comprises means for altering the distance between the first container and the second container so as to selectively provide a first relative distance between the first container and the second container wherein the first chamber and the second chamber (enclosed by the respective first container and second container are fluidly separated and a second relatively closer distance between the first container and the second container wherein the first chamber and the second chamber are in fluid communication.

The means for altering the distance between the first container and the second container preferably comprise a helical member, in particular a helical grove that is configured to translate a rotation into an axial movement of one container with respect to the other container.

In a preferred embodiment, the first container is held in the housing by a releasable snap fit connection that is released if a force exerted by the second container on the first container exceeds a predetermined threshold.

A second aspect of the invention is a set of at least two distinct containers that can be combined to form a single, fluid tight assembly. The first chamber that is enclosed by the first container comprises a first set of chemicals and/or agents. Preferably, the first chamber comprises a fluid for lysing a sample. Accordingly, the first container preferably is a lysis container and the first chamber is a lysis chamber. The first chamber is closed prior to use. The second chamber that is enclosed by the second container comprises a second set of chemicals and/or agents that are at least in part distinct from the chemicals and/or agents of the first set of chemicals in the first chamber. Preferably, the second set of chemicals comprises chemicals required for testing for an analyte and in particular to amplify nucleic acids in a sample. Accordingly, the second container preferably is a test container enclosing a test chamber. The first container comprises an interface, in particular a lid, that can be opened when the first container and the second container are combined to form a single, fluid tight assembly, in order to allow contents of the first chamber to enter the second chamber.

The second aspect of the invention can implemented in combination with the first aspect of the invention or independently from the first aspect of the invention.

In any embodiment, the test container may comprise more than one, e.g, two, three or four separate test chambers. In each test chamber a different mixture of chemicals can be contained thus allowing for testing for different target analytes simultaneously.

Preferably each container is an integral unibody. In an alternative embodiment, the assembly of the first container and the second container is an integral unibody.

Preferably, the containers can be connected by means of a fluid tight snap fit connection to thus form a single, fluid tight assembly. As an alternative to a snap fit connection, a screw lock connection similar to a Luer-lock can be provided for tightly connecting the containers. Another alternative is a press fit connection between the containers.

The advantage of a fluid tight assembly of at least two containers is that the assembly can be disposed easily without the risk of contamination because the contents of the assembly is tightly secured within the assembly.

Preferably, the second container has transparent walls that allow excitation and detection of luminescence and in particular fluorescence.

An assembly comprising two initially separate containers and a fluorescence detection device with a receptacle that can receive the assembly for luminescence detection makes it possible to perform a testing method that comprises two consecutive chemical or biochemical method steps—for instance lysis and amplification—and a luminescence testing step in a simple, clean and safe manner that minimizes the risk of contamination and infection while providing easy handling. The fluorescence detection device can be re-used because in use it is not contacted by the sample or any agents or chemicals since these are tightly enclosed in the assembly of containers. The assembly of containers and its contents can be safely disposed after use because the contents is reliably kept within the interior of the assembly.

In a preferred embodiment, the system also comprises a piston for transferring fluid from the first chamber to the second chamber. The piston is configured to fit into the first chamber. Preferably, the travel of the piston is limited and corresponds to the amount of fluid to be transferred into the second chamber. The piston can comprise a lid for closing the lysis container and the test container.

The combination of the lysis container and piston is arranged such that in the event of the piston moving into the lysis container pressure release is possible through venting. The venting means are configured to prevent content of either the lysis container or the test container or both from escaping out of the respective chamber or chambers.

The first containers lid that allows fluid transfer from the first chamber to the second chamber, when the first container and the second container are connected, can be configured to be opened by connecting the first container and the second container.

According to a further aspect, a set of containers is provided, comprising at least one test container and a sample container that is configured to for collecting a fluid sample. In particular, the test container is configured for collecting a sample by way of gargling or mouth washing. By such embodiment, sampling by means of a swab is replaced by a test using mouthwash or gargling. This embodiment follows the basic concept as disclosed herein before featuring modified parts so that a simple testing can be achieved by mouthwashing and/or gargling. The reliability of the samples and subsequent test process is improved and the inconveniences of sample taking with a swab are avoided.

The set of containers preferably comprises a sample container, a lysis container and a test container.

Alternatively, the set of containers comprises a sample container that comprises a sample chamber and a compartment for a lysing fluid, said compartment initially being separated from the sample chamber. In such embodiment, no separate lysis container is needed.

In yet another embodiment, the sample chamber of the sample container can comprise a liquid that can be used for gargling and that can lyse a sample thus acquired.

The set of container is part of a system that further comprises a fluorescence detection device.

The system is configured to implement an isothermal amplification method such as RPA and SIBA and other methods, preferably isothermal methods. The amplification method is configured to be carried out in a temperature range between 25° C. and 47° C. In various embodiments, initial heating may be applied. Other embodiments implement continuous heating. There are also embodiments without any external heating. in one or more of the method steps.

The fluorescence detection device comprises
a detection chamber configured to receive the second container, i.e. the test container or the contents of the test container,
a light source,
an optical sensor
an energy supply
a wireless data interface and
a controller.

The controller can be a microcontroller and/or a state machine.

The fluorescence detection device may further comprise heating means that allow heating of a test container that is inserted in the fluorescence detection device.

Preferably, the detection device and the second container comprise mutually engaging features that prevent a relative rotation between the detection device and the second container.

The detection chamber and the receptacle preferably are arranged in a truncated cone shaped protrusion of a housing of the detection device. The shape of sad preferably matches an inner shape of a flared section of a housing of a fluid tight assembly that includes at least the first and the second container.

The system can either be a point of care (POC) system wherein the fluorescence detection device is arranged at a point of care, for instance in a medical doctors office. Alternatively, the system may be a personal system wherein the fluorescence detection device is self-contained and mobile, in particular pocketable.

The invention shall now further be illustrated by way of an example and with a reference to the figures. Of the figures,

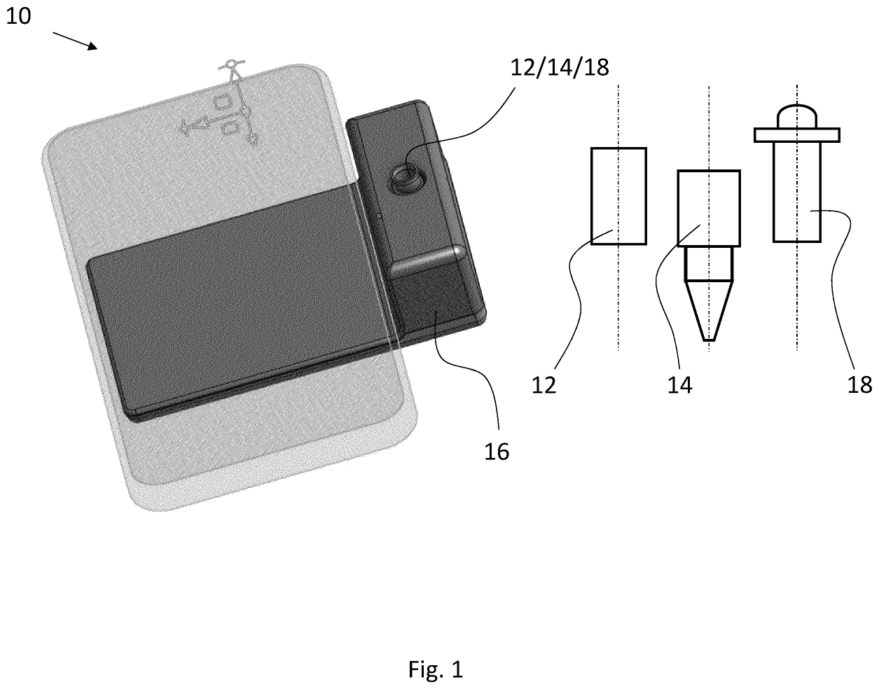

FIG. 1: shows a detection system for detecting a target analyte by way of isothermal nucleic acid amplification and fluorescence;

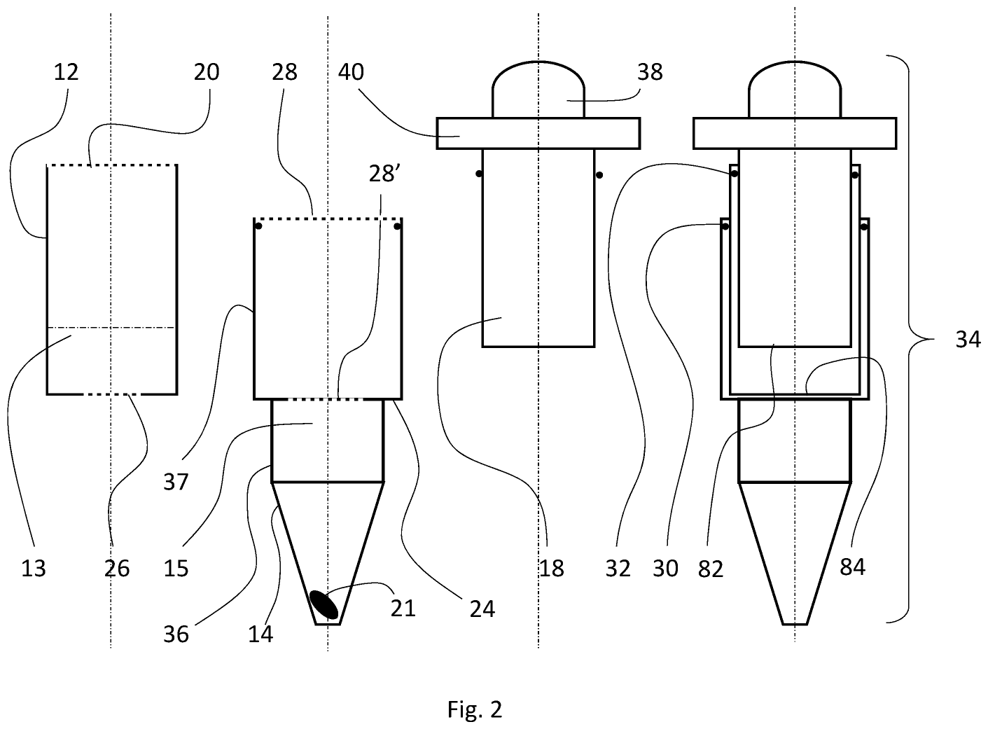

FIG. 2: shows a first embodiment of a set of containers to be used with the detection system of FIG. 1;

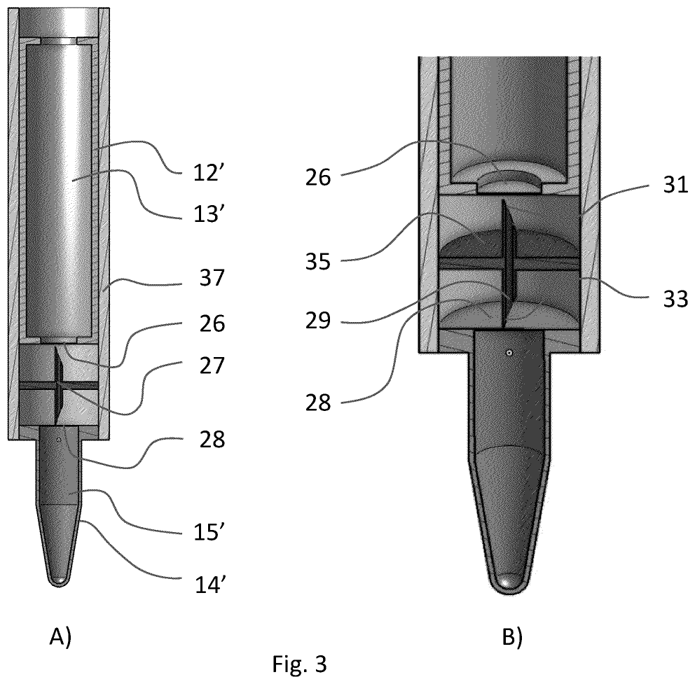

FIG. 3a,b: show a piercing fluid transfer interface to establish a fluid connection between the lysis chamber and the test chamber;

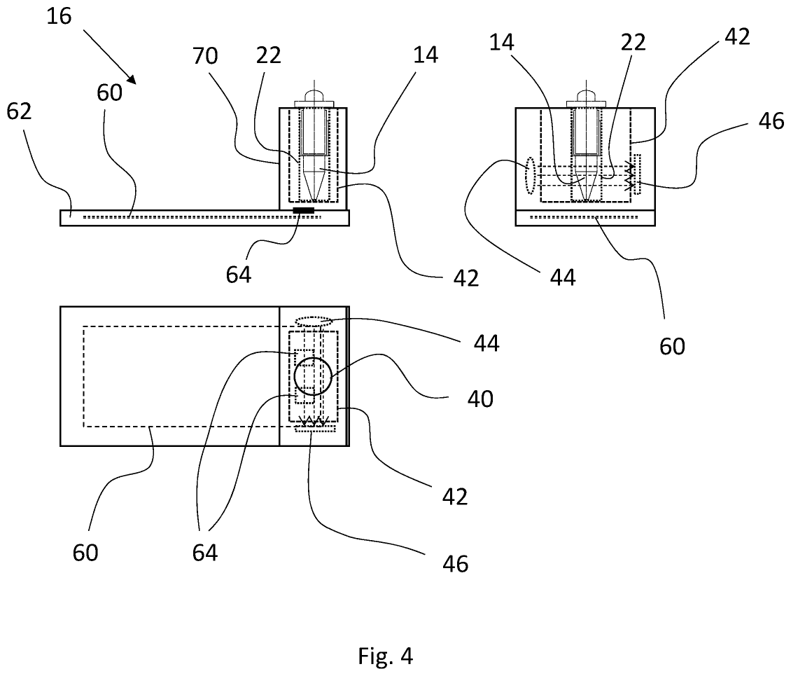

FIG. 4: shows schematic diagrams of a detection device of the system from FIG. 1;

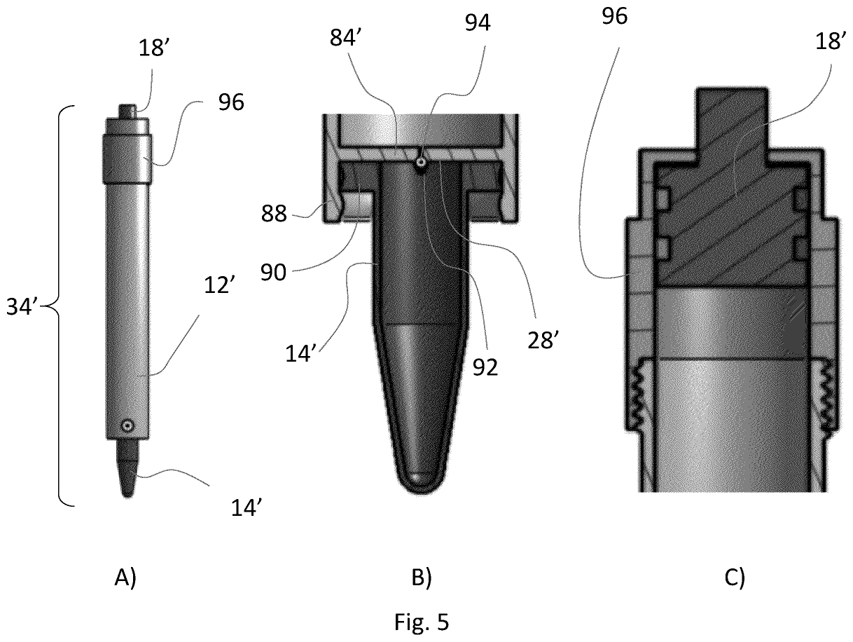

FIG. 5a-c: show a second embodiment of a set of containers to be used with the detection system of FIG. 1;

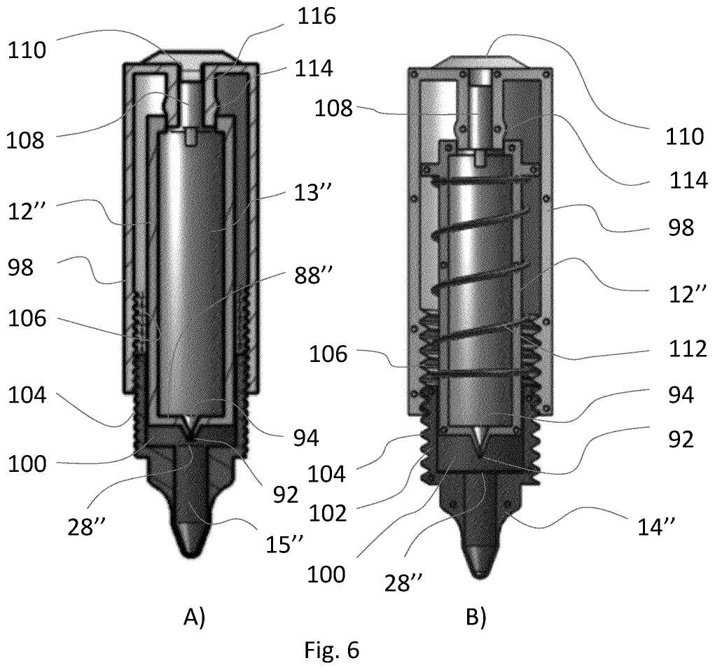

FIG. 6a,b: show a third embodiment of a set of containers to be used with the detection system of FIG. 1;

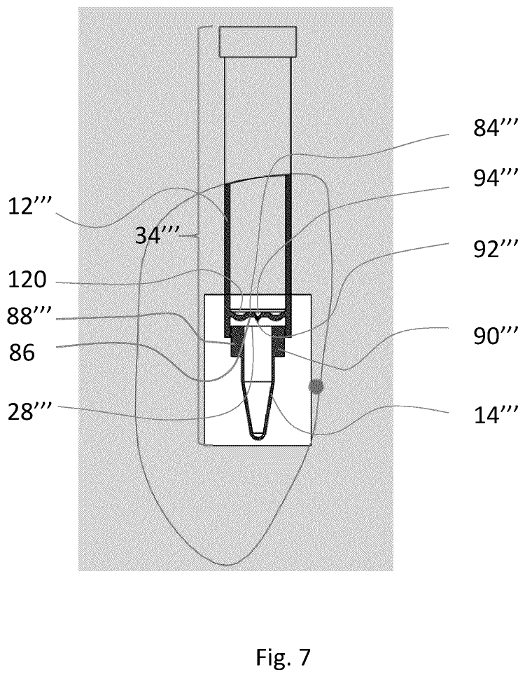

FIG. 7: shows a fourth embodiment of a set of containers to be used with the detection system of FIG. 1;

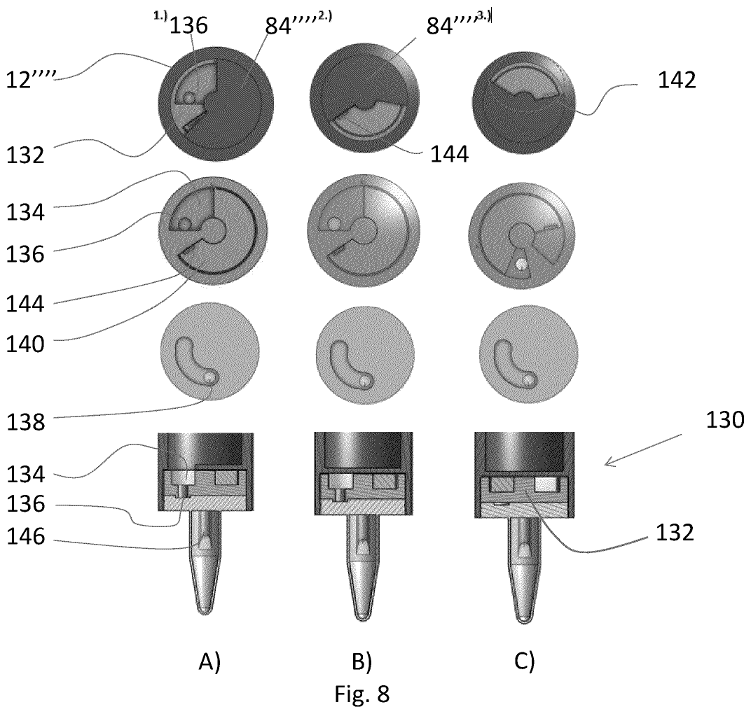

FIG. 8a-c: show a fifth embodiment of a set of containers to be used with the detection system of FIG. 1;

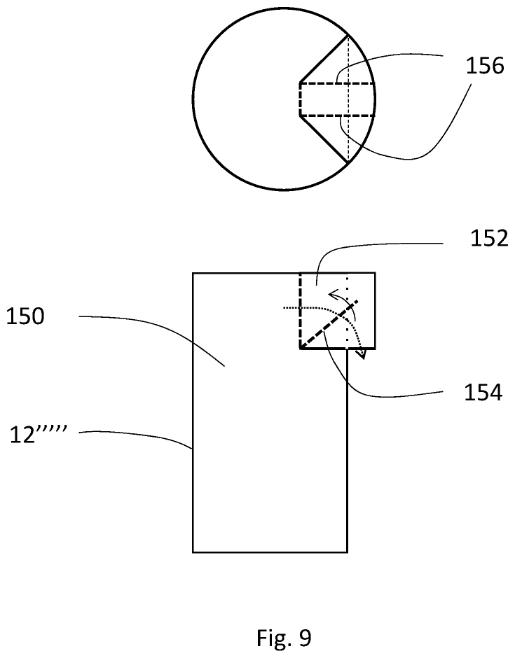

FIG. 9: shows a sixth embodiment of a set of containers to be used with the detection system of FIG. 1;

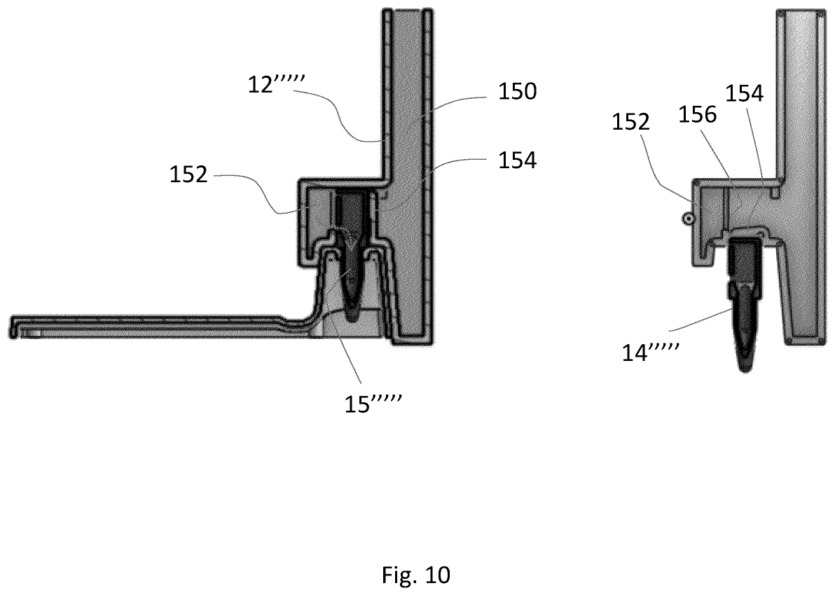

FIG. 10: shows a seventh embodiment of a set of containers to be used with the detection system of FIG. 1:

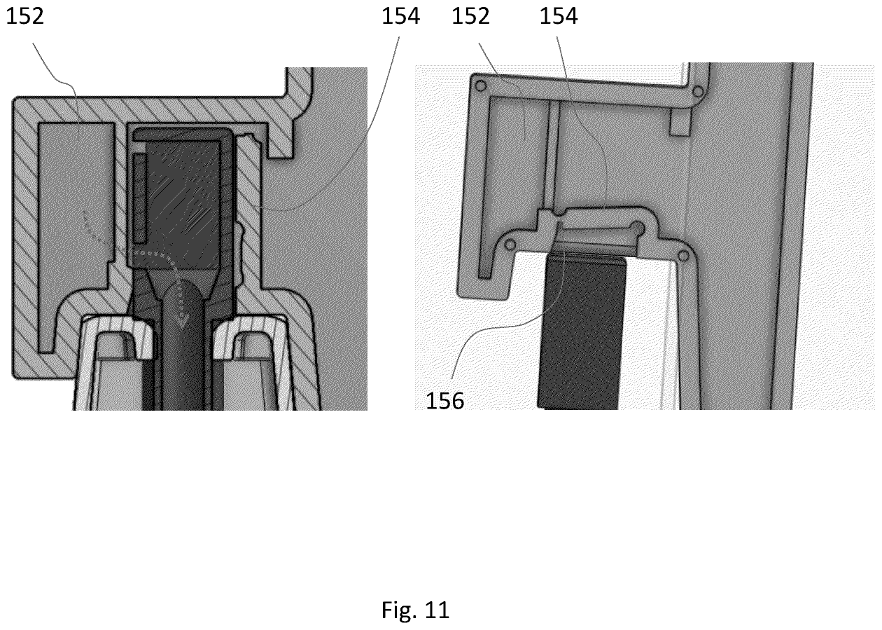

FIG. 11: details of the seventh embodiment of FIG. 10;

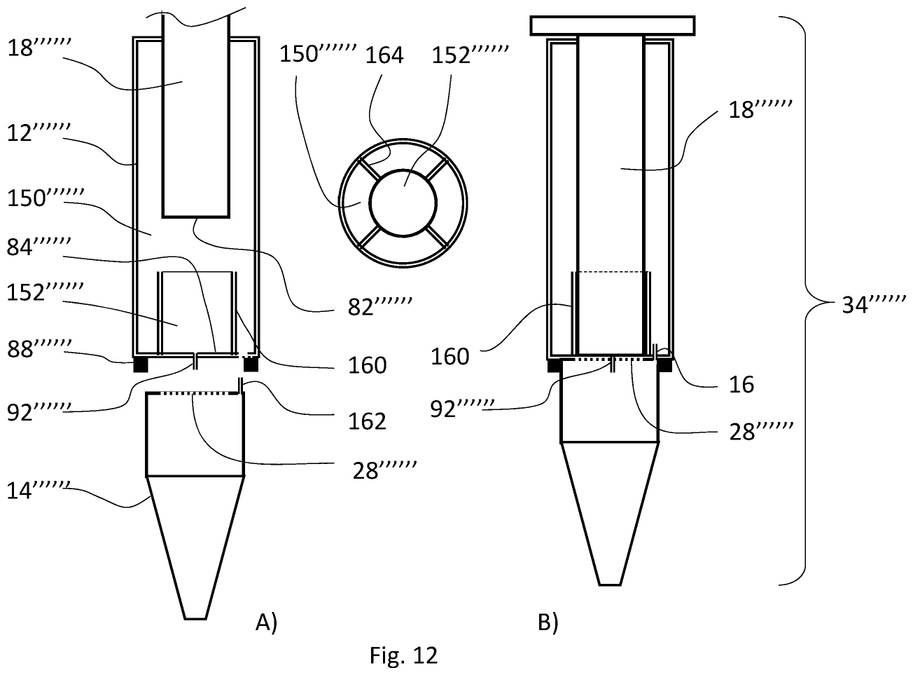

FIG. 12a, b: show an eighth embodiment of a set of containers to be used with the detection system of FIG. 1; and

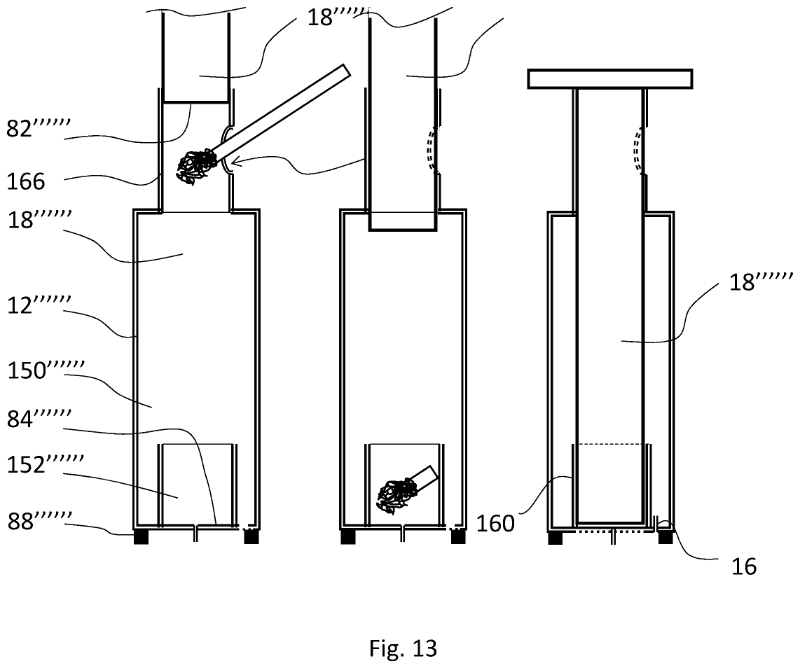

FIG. 13: shows a lysis chamber with cutting means for cutting a test swab;

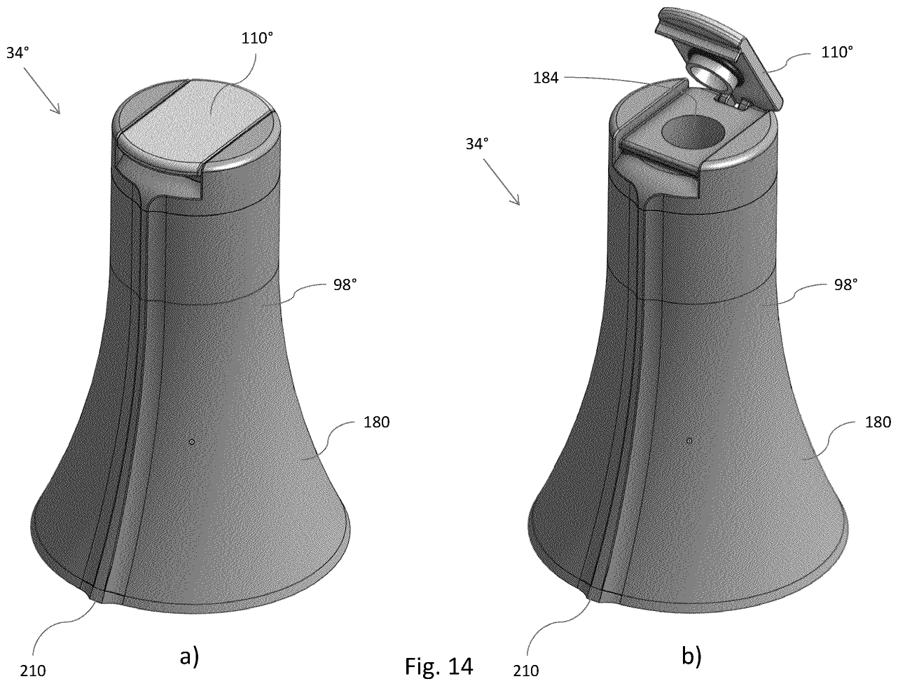

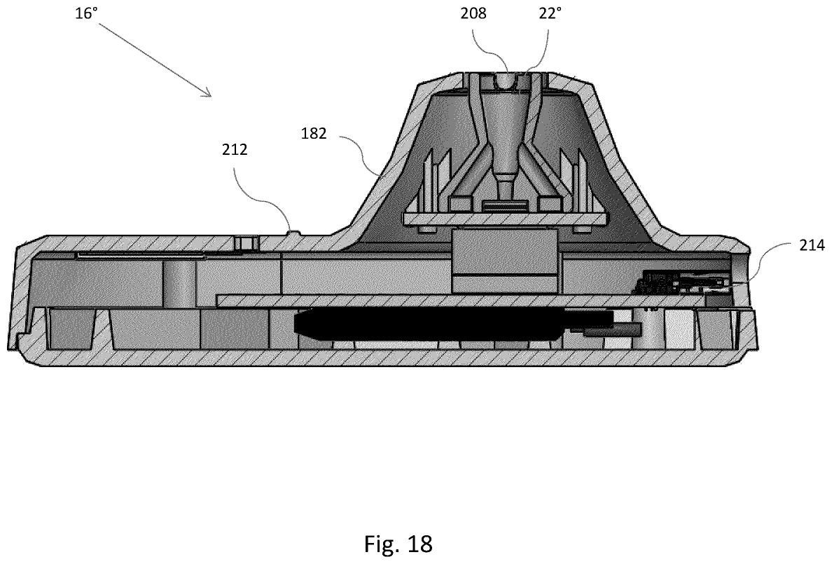

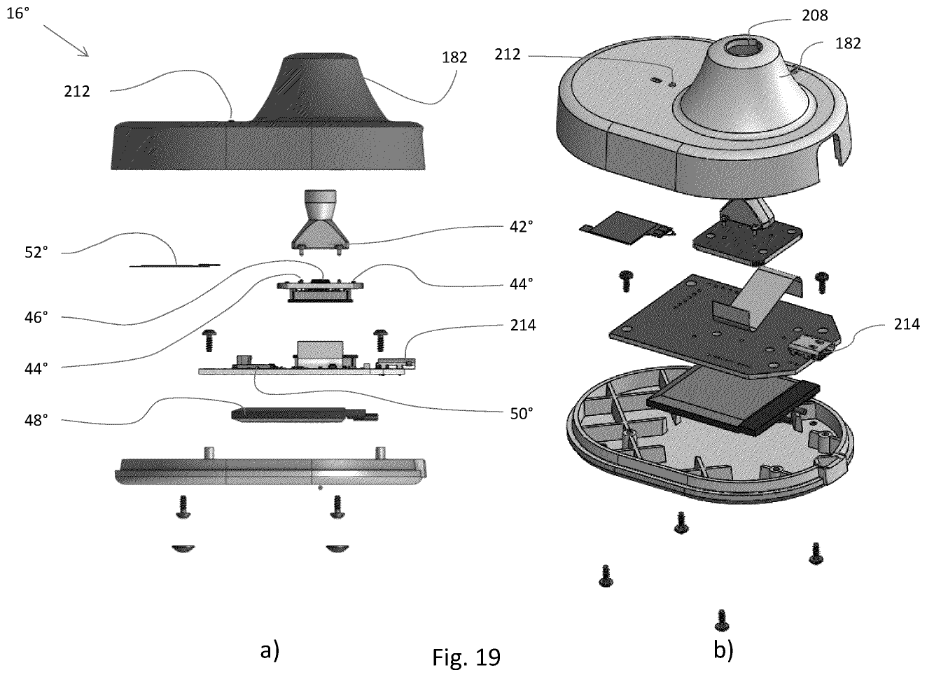

FIG. 14a: is an isometric view of a further embodiment of a set of containers forming a fluid tight assembly to be used with the detection system of FIG. 1 or the detection device of FIGS. 18 and 19;

FIG. 14b: is an isometric view of the fluid tight assembly from FIG. 14a with an open lid;

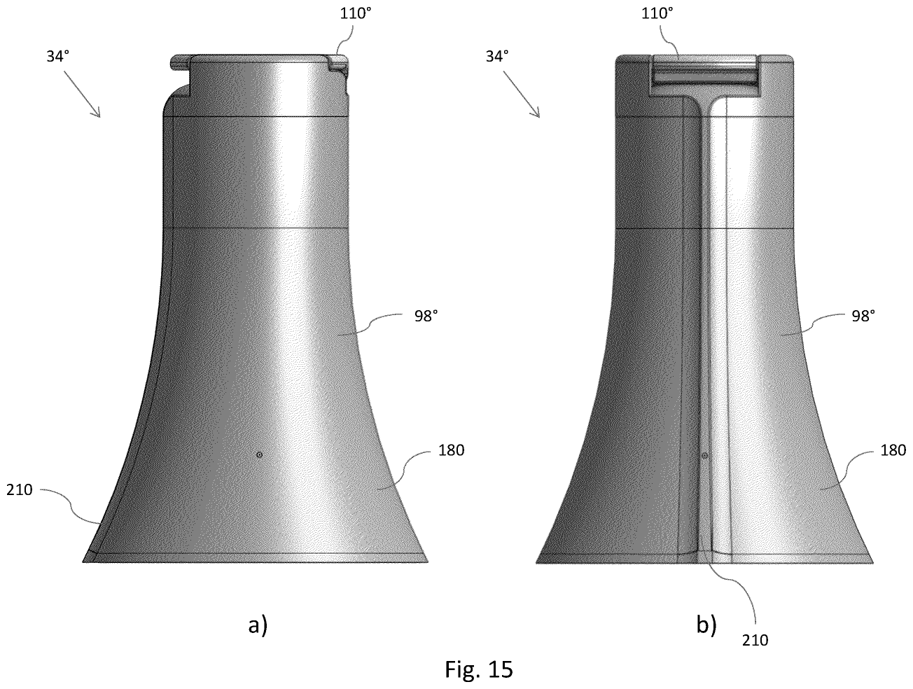

FIG. 15a: is a front elevation view of the fluid tight assembly from FIG. 14;

FIG. 15b: is side elevation view of the fluid tight assembly from FIG. 14;

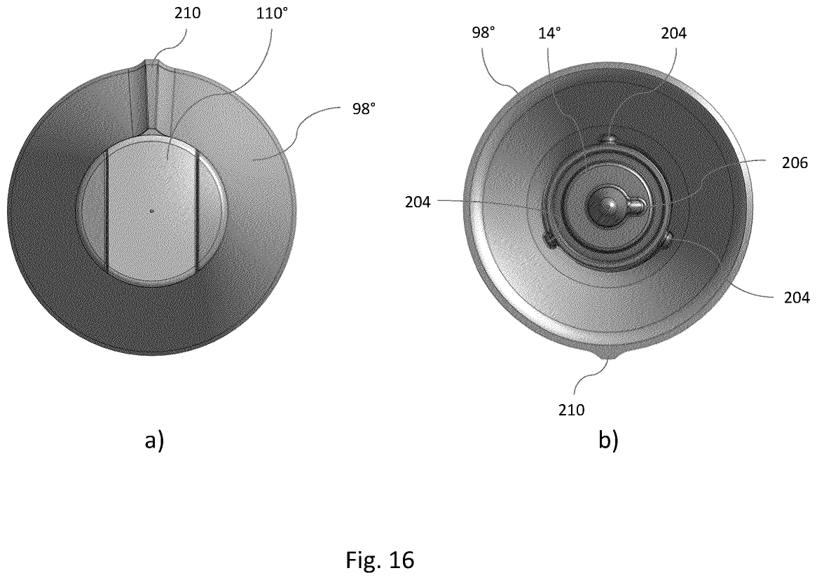

FIG. 16a: is a top view of the fluid tight assembly from FIG. 14;

FIG. 16b: is a bottom view of the fluid tight assembly from FIG. 14;

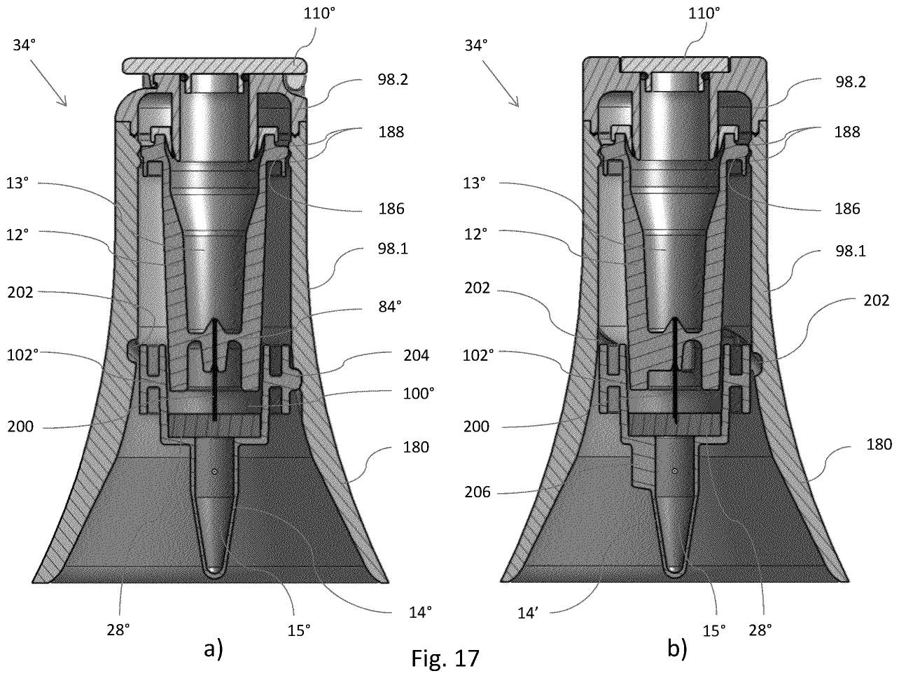

FIG. 17a: is a cross sectional view of the fluid tight assembly from FIG. 15a;

FIG. 17b is a cross sectional view of the fluid tight assembly from FIG. 15b;

FIG. 18: is a cross sectional view of a testing device adapted to match the fluid tight assembly from FIGS. 14 to 17; and

FIG. 19a: is an exploded side view of the testing device of FIG. 18;

FIG. 19b: is an exploded isometric view of the testing device of FIG. 18; and

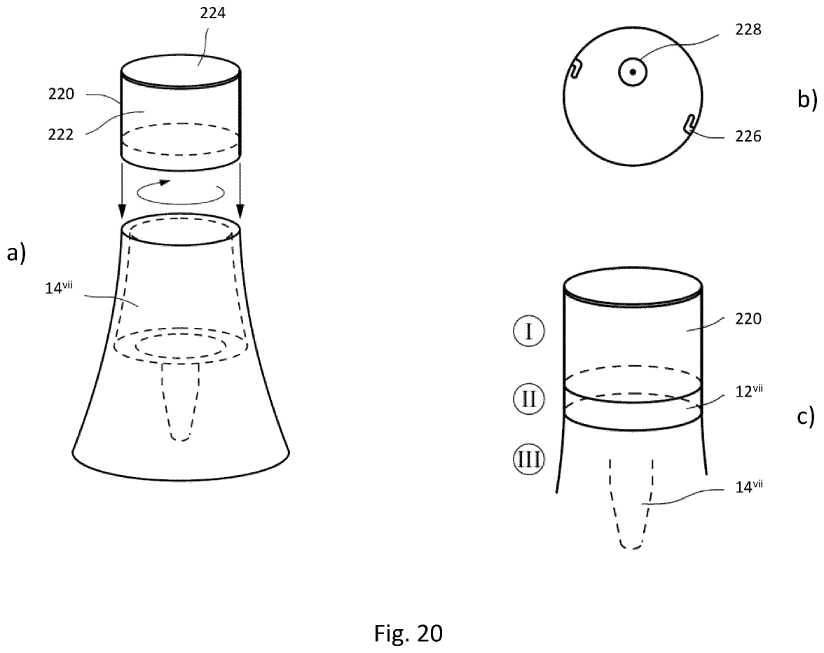

FIG. 20a-c: show an alternative embodiment having a third container for collecting a fluid sample.

The system 10 for detecting a target analyte comprises a fluorescence detection device 16 and a set of two containers 12 and 14. A first container 12 is a lysis container that encloses a first chamber 13 comprising a fluid and/or an agent for lysing a sample. The second container 14 is a test container 14 that encloses a second chamber 15 comprising reagents, for instance enzymes for amplifying nucleic acids in a sample that was lysed in the lysis container 12; cf. FIG. 1.

The set of two containers 12 and 14 further comprises means that allow a limited transfer of fluid from the first chamber 13 into the second chamber 15. In the embodiment shown in FIGS. 1 and 2, the means for limited transfer of fluid from the first container 12 to the second container 14 comprise a piston 18.

The lysis container 12 contains a lysing fluid that causes lysing of cells or viruses in a sample to be tested. By way of lysing, nucleic acids such as DNA or RNA are released by way of breaking down the cells or viruses in the sample to be tested. Lysing can be achieved by a lysing fluid that comprises an acid such as hydrochloric acid or a weak alkali.

The lysis container 12 has a lid 20 so lysis container 12 can be opened and a sample to be tested can be entered in the lysis container 12; cf. FIG. 2. The contents of the lysis container 12 is about 100 μl.

Lid 20 of the lysis container 12 preferably is a membrane 20 that can be pierced by a cotton swab with a sample. The membrane 20 can be a composite film, for instance made from plastic sheeted aluminum. Alternatively, the membrane 20 can be an integral part of the lysis container 12, in particular a thin septum formed from the material of the lysis container walls. Lid 20 can alternatively be a cap that can be opened and closed.

The test container 14 encloses the second chamber 15 comprising a mixture of enzymes that are needed for a recombinase polymerase amplification (RPA). Preferably, the mixture is provided in the form of a dry pellet 21 that is contained in the second chamber.

The test container 14 preferably is a cuvette that can be inserted in a receptacle 22 of the fluorescence detection device 16. The receptacle 22 is part of a detection chamber 42 of the fluorescence detection device 16.

The test container 14 is dimensioned to allow inserting the lysis container 12 into the test container 14 (or vice versa) in a tight and defined manner. An abutment 24 limits the depth of insertion. Once the lysis container 12 is fully inserted into the test container 14, the piston 18 can be used to transfer the fluid from the lysis container into the test container 14 and allowing the recombinase polymerase amplification to work in the closed test container 14. To allow the transfer of the contents of the lysis container 12 into the test container 14, a further thin lid 26 is arranged as a septum at the bottom of the lysis container 12. The thin lid 26 is dimensioned to break under the fluid pressure caused by the piston. Alternatively, the piston 18 and the thin lid 26 can be designed so that a tip (not shown) of the piston 18 can pierce the thin lid 26.

The test container 14 may also be closed by a septum, for instance a thin pierceable lid 28 or 28′. The pierceable lid 28 maybe arranged at the otherwise open end of a guiding sleeve 37 of test chamber 37 or close to the level of the abutment 24 (lid 28).

The thin lids 26 and 28 can be a composite film, for instance made from plastic sheeted aluminum. Alternatively, the thin lid 26 can be an integral part of the lysis container 12, in particular a thin septum formed from the material of the lysis containers 12 walls. Further preferred materials for the thin lids 26 and 28 are silicone or a thermoplastic elastomer (TPE).

In order to create a fluid interface and establish a fluid connection between lysis container 12 and test container 14, a piercing fluid transfer interface member 27 is provided; see FIG. 3. The piercing fluid transfer interface member 27 comprises a fluid passageway 29 formed in tube-shaped protrusions 31 and 33 that extend centrally from a support disc 35 in opposite directions. Each tube-shaped protrusion 31 and 33 has a pointed tip that can pierce lid 26 and 28, respectively. The tube-shaped protrusions 31 and 33 maybe formed from a metal tube that extends through the support disc 35. Alternatively, tube-shaped protrusions 31 and 33 maybe integrally formed with the support disc 35 from a plastic material.

Lysis container 12, test container 14 and piercing fluid transfer interface member 27 are held and guided in the guiding sleeve 37. The fluid passageway 29 extends centrally along a common longitudinal axis of the lysis container 12, the test container 14 and the guiding sleeve 37. Piercing fluid transfer interface member 27 could be fixed to guiding sleeve 37 or even an integral part of guiding sleeve 35 so that lysis chamber 12 and test chamber 14 could be inserted into guiding sleeve 35 until respective lid 26 or 28 is pierced. However, it is preferred if piercing fluid transfer interface member 27 can slide within guiding sleeve 37 because this would provide that both lids 26 and 28 are pierced simultaneously. This is, because a sliding piercing fluid transfer interface member 27 would be pushed by either lid 26 or 28 until piercing fluid transfer interface member 27 abuts the respective other lid 28 or 26. Only when sliding fluid transfer interface member 27 touches both lids 26 and 28, piercing of the lids would occur.

Test container 14 and guiding sleeve 37 could form an integral unibody similar to what is shown in FIG. 2. If the test container 14 and the guiding sleeve 37 are firmly connected so as to form an integral part, it is mandatory that the fluid transfer interface member 27 is slidingly arranged within guiding sleeve 37. FIG. 3B shows an enlarged detail from FIG. 3B in semi-perspective view.

The lysis container 12, the test container 14 and the piston 18 are configured to engage in a fluid-tight manner when fully inserted. Such fluid tight engagement can be achieved by means of a snap fit connection wherein an annular protrusion 30 of one of the lysis container 12 and the test container 14 engages in an annular groove of the respective other container. Likewise, an annular protrusion 32 of one of the piston 18 and the lysis container 12 engages in an annular groove of the respective other part. Annular protrusions 30 and 32 act as sealings and may be integrally formed with the rest of the respective container or piston.

As an alternative to a snap fit connection, a screw lock connection similar to a Luer-lock can be provided for tightly connecting the lysis container 12 and the test container 14.

To allow insertion of the lysis container 12 into the test container 14 and of the piston 18 into the lysis container 12, venting means (not shown) are provided.

Once fully engaged, the test container 14, the lysis container 12 and the piston 18 form a tight assembly 34 that can be handled as a single, fluid tight unit.

Walls 36 of the test container 14 are transparent so as to allow light to enter the test container 14 and to exit the test container 14. The transparent walls 36 of the test container 14 make it possible to expose the content of test container 14 to exiting light that can cause a luminescence. In case, the content of the test container 14 is luminescent luminescence of the sample in the test container 14 can be detected through the transparent walls 36 of the test container 14.

The pellet 21 that contains the mixture of enzymes for recombinase polymerase amplification preferably comprises a recombinase, a single-stranded DNA-binding protein (SSB) and a strand-displacing polymerase, exonuclease III and in case RNA is to be detected, a reverse transcriptase.

In use, a sample to be tested first is filled into lysis container 12. After lysing the cells in the sample to be tested, the entire content of lysis container 12 is transferred into the test container 14 by means of the piston 18 so that a recombinase polymerase amplification can occur in the test container 14.

Once the recombinase polymerase amplification has occurred in the test container 14—typically between 10 to 15 minutes after filling in the content of the lysis container 12 into the second chamber that is enclosed by the test container 14—the test container 14 or alternatively only its content can be entered into the fluorescence detection device 18.

In the illustrated, preferred embodiment, the entire assembly 34 is inserted in a receptacle 22 of the fluorescence detection device 16; cf. FIG. 4. For handling of the assembly 34, a grip 38 is provided at a proximal end of the piston 18.

In order to prevent external light, for instance stray light, from entering into the receptacle once the assembly 34 is fully inserted in the receptacle 22, a collar 40 is provided that forms a lid for the receptacle 22.

The fluorescence detection device 16 comprises a detection chamber 42 that is configured to receive the test container 14 or the contents of the test container 14. In the illustrated, preferred embodiment, the receptacle 22 is part of the detection chamber 42. Within the detection chamber 42 or adjacent to the detection chamber 42, a light source 44 and an optical sensor 46 are arranged. The light source 44 is configured to illuminate the contents of the detection chamber 42 with a light that can cause luminescence in a sample to be tested during and after the sample has undergone recombinase polymerase amplification. The optical sensor 46 is arranged and configured to detect luminescence in the detection chamber 42 in case luminescence occurs.

The detection chamber 42 is arranged in a detection chamber housing 70 that has outer dimension smaller than 10 cm by 10 cm by 4 cm. Preferably the volume of the entire fluorescence detection device 16 is smaller than 200 cm², and even more preferred smaller than 100 cm². In a preferred embodiment, the longest outer dimension is a least twice as long as the shortest outer dimension.

The fluorescence detection device 16′ may provide a second receptacle wherein the lysis container 12 can be placed prior to the connection with the test container 14. The second receptacle can also host the test container 14 and the lysis container 12 prior to use.

With respect to the lysis container 12 and the test container 14 it is an object to only transfer a limited amount of fluid from the lysis container 12 to the test container 14. Preferably, the amount to be transferred from the lysis container 12 into the test container 14 is reproducible within a range of tolerance of ±20% of the transferred volume. For instance, it is an object to transfer about 50 μl from the lysis container 12 into test container 14, while the total amount of liquid in lysis container 12 is larger, for instance more than 100 μl.

In FIG. 2, a level 80 of fluid is shown. Piston 18 is configured to only displace about 50 μl of the fluid in lysis container 12. This is achieved by a piston 18 that is shorter than the length of the first chamber that is enclosed by the lysis container 12 so that bottom 82 of piston 18 will not reach the bottom 84 of the first chamber even if the piston 18 is fully introduced into the lysis container 12. Thus, only a reproducible share of the fluid in lysis container 12 is transferred into the test container 14. The transferred share of fluid may for example correspond to 50 μl of fluid.

An implementation of an assembly 34′ similar to the assembly 34 of FIG. 2 is shown in FIGS. 5a, b and c. Test container 14′ can be connected to lysis container 12′ by a snap fit connection. The cylindrical side wall of the lysis container 12 extends beyond a bottom 84′ and thus forms an extension 88. Test container 14′ has an annular flange 90 that allows for a snap fit connection with the lysis container 12′ when the flange 90 engages with the extension 88; see FIG. 5b.

Test container 14′ is closed by a lid 28′ that is sealingly connected to the annular flange 90 of test container 14. Lid 28′ preferably is made from an annular composite film. Alternatively, the thin lid 28′ can be an integral part of the test container 14, in particular a thin septum formed from the material of the test containers 14 walls.

In the center of bottom 84′ of lysis container 12′, a pointed central tip 92 is arranged and a valve 94 extends through the central pointed tip 92. The central pointed tip 92 maybe made from metal or from plastic material.

A piston 18′ with limited travel is integrated in a screw-on cap 96. Screw-on cap 96 that can be screwed on lysis container 12′. Accordingly, a swab with a sample can be inserted into the first chamber that is enclosed by a lysis container 12′ and thereafter, lysis container 12′ can be closed by screwing on the screw-on cap 96. Once lysis chamber 12′ is closed with the screw-on cap 96, lysis container 12′ can be connected to test container 14′. When test container 14′ snaps into a lysis container 12′, the pointed tip 92 at the center of bottom 88′ of lysis container 12′ pierces into lid 28′. Then, piston 18′ can be pushed to thus press a limit amount of fluid from the first chamber enclosed by the lysis container 12′ into the second chamber that is enclosed by the test container 14′.

In yet another embodiment as shown in FIGS. 6a and b, lysis container 12″ having a bottom 88″ with a pointed central tip 92 and a valve 94 arranged therein can be inserted into a housing 98. Lysis chamber 12″ as an outer diameter that fits into a lysis container receptacle 100 that is formed by a test container extension 102. Test container extension 102 is provided with a threating 104 on the outside of the test container extension 102.

The housing 98 is provided with an inner threating 106 that fits to the outer threating 104 of the test container 14″.

When the lysis chamber 12″ is inserted into the housing 98, housing 98 can be screwed on the test container 14″ to thus press the lysis container 12″ into the lysis container receptacle 100 of the test container 14″. Once the pointed central tip 92 at the bottom 88′ of lysis container 12″ pierces the lid 28′ that initially closed the second chamber in the test container 14″, fluid can be transferred from the lysis container 12″ into the test container 14″ by further compressing the lysis container 12″. Further rotating of housing 98 causes further compression of the lysis container 12″ until an annular opening of the lysis container 14″ overcomes an annular protrusion 114 on a central extension 116 of housing 98 extending into the lysis container 12″. Compression of the lysis container 12″ is thus limited by the force needed for the annular opening of the lysis container 14″ to overcome the annular protrusion 114.

Prior to piercing lid 28″, a swab with a sample can be inserted into lysis container 12″ to have the sample lysed by the lysing fluid in the lysis container 12″. A central opening 108 in the central extension 116 of the housing 98 is provided that allows insertion of a swab into the first chamber that is enclosed by the lysis container 12″. The central opening 108 can be closed by a push-on cap 110. In order to prevent the pointed tip 92″ from prematurely piercing the lid 28″ of test container 14″, a helical spring 112 can be provided, that pushes the lysis container 12″ away from the test container 14″. The spring force of spring 112 is overcome by turning housing 98 and thus screwing housing 98 further onto the test container 14″.

Instead of arranging the central pointed tip on the bottom of lysis container 12″, a central pointed tip could be arranged at the top of the test container instead of lid 28″. In such embodiment, a pierceable lid would be arranged at the bottom of the lysis container.

An alternative approach is provided by an assembly 34′″ as is illustrated in FIG. 7. The assembly 34′ also comprises a lysis container 12′″ and a test container 14′″. At the bottom 84′″ of lysis container 12′″ an elastic valve 86 is arranged that opens, when the test container 14′″ is inserted into an extension 88′″ of lysis container 12′″.

Initially, test container 14′″ is closed by a lid 28′″ that is made from an aluminum composite film. The chamber enclosed by test container is evacuated so that a vacuum exists within the test container 14′. The lid 28′″ of test container 14′″ is sealingly connected to an annular flange 90′″ of test container 14′″.

When the test container 14′″ is inserted into the extension 88′″ of the lysis container 12′″, the annular flange 90′″ will abut an annular, convex bulge 120 of valve 86 at the bottom 84″″ of the lysis container 12′″. This will cause a pointed center part 92′″ of valve 86 to pierce into the aluminum composite film 28′″ covering the test container 14′″. At the same time, a central hole 94′″ in the pointed central tip 92′″ of valve 86 will let a definite amount of fluid flow from the lysis container 12′″ into the test container 14″″. This is because initially, a vacuum exists within the test container 14′″. Elastic forces of the elastic valve 86 will keep a central opening 94′″ in the pointed central tip 92′″ of valve 86 closed until the annular bulge 120 of valve 86 is compressed in an axial direction of the lysis container 12′″ due to the abutting annular flange 90′″ of the test container 14′″.

In FIGS. 8a, b and c a fluid transfer interface of yet another embodiment of an assembly comprising a lysis container 12″″ and a test container 14″″ is illustrated. At the interface between the lysis container 12″″ and the test container 14″″ a portioner 130 is arranged. Portioner 130 comprises a base member 132 having an annular groove 134 facing towards the first chamber that is enclosed by the lysis container 12″″. The annular groove 134 extends about 3400 to 350°. At one end of the annular groove 134 a through-hole 136 is provided that can be brought in alignment with an opening 138 being an fluid communication with the second chamber that is enclosed by the test container 14″″. A slide 140 is provided that can rotate with respect to the base member 132 to thus displace fluid in the annular groove 134.

FIGS. 8A to 8C illustrate the operation of the portioner 130. In the initial position, an annular segment shaped opening 142 in the bottom 84″″ of the lysis container 12″″ is in fluid communication to the annular groove 134 so that fluid from the first chamber that is enclosed by the lysis container 12″″ can flow into the annular groove 134. Rotating the lysis container 12″″ with respect to the portioner 130 closes the fluid passage between the first chamber and the annular groove 134; see FIG. 8B. The annular segment shaped opening 142 has moved due to rotation so that the annular groove 134 in the portioner 130 is closed.

An edge 144 of the annular segment shaped opening 142 engages an attachment 144 of slide 140 so that the slide 140 rotates together with the lysis container 12″″. Rotation of slide 140 causes also the base member 132 of portioner 130 to rotate because the fluid in the annular groove 134 cannot be compressed. Rotation of the base member 132 of portioner 130 occurs, until the through-hole 136 in the bottom of the annular groove 134 is aligned with the opening 138 of the test container 14″″ that is in fluid communication with the second chamber that is enclosed by the test container 14″″; cf. FIG. 8C.

A radial extension 146 of the test container 14″″ can engage with a groove in the receptacle 22 of the detection chamber 42 of the fluorescence detection device 16. The radial extension 146 prevents the test container 14″″ from rotating.

In yet another embodiment, as shown in FIGS. 9 to 11, the lysis container 12′″″ may comprise the first chamber 150 and a third chamber 152 and a movable separation member 154. The movable separation member 154 is configured to be moved between a first state and a second state. In the first state, the movable separation member 154 allows fluid to pass from the first chamber 150 to the third chamber 152. In the second state, movable separation member 154 blocks fluid from passing from the first chamber 150 to the third chamber 152. The movable separation member 154 preferably is an integral wall part of lysis container 12′″″ that is connected to a wall of lysis container 12′″″ by a breaking score line 156. Breaking of the score line 156 occurs, when the lysis container 12′″″ is connected with the test container 14′″″ and causes the movable separation member 154 from moving from the first state to the second state. The third chamber 154 that is enclosed by the lysis container 12′″″ encloses a volume that corresponds to the volume of liquid that is to be transferred from the first chamber in the lysis container 12′″″ to the second chamber in the test container 14′″″.

When the lysis container 12′″″ and the test container 14′″″ are connected, a fluid passage is established between the third chamber 152 in the lysis container 12′″″ and the second chamber in the test container 14′″″ thus allowing a definite amount of fluid flowing from the third chamber 152 into the second chamber.

In yet another embodiment of assembly 34″″″ comprising a lysis container 12″″″ and a test container 14″″″ as shown in FIGS. 12a and b, lysis container 12″″″ encloses a first chamber 150″″″ and a third chamber 152″″″. The third chamber 152″″″ is defined by a cylindrical side wall 160 and by the bottom 84″″″ of the lysis container 12″″″. The end of the third chamber 151″″″ that faces away from the bottom 84′″ is open so that the first chamber 150″″″ and the third chamber 152″″″ are not separated. The inner diameter of the third chamber 152″″″ fits to an outer diameter of a piston 18″″″ that thus can be inserted into the third chamber 152″″″. Once a bottom 82″″″ of piston 18″″″ engages with the side wall 160 of the third chamber 152″″″, the first chamber 150″″″ and the third 152″″″ are fluidly separated from one another. The volume enclosed by the side wall 160 is about 50 μl to 70 μl.

In order to transfer fluid from the lysis container 12″″″ to the test container 14″″″, test container 14″″″ can be attached to the bottom 84″″″ of the lysis container 12″″″ and will be held by an extension 88″″″ of the lysis container 12″″″. Once attached, a pointed central tip 92″″″ pierces into a lid 28″″″ of the test container 14″″″. Further, a venting tip 162 pierces into the bottom 84″″″ of the lysis chamber 12″″″ outside of the third chamber 152″″″. This is shown in FIG. 12B.

In order to transfer fluid from the lysis chamber 12″″″ into the test chamber 14″″″, the piston 18″″″ is pushed into the lysis chamber 12″″″ and will not cause any fluid transfer from the lysis container 12″″″ to the test container 14″″″ until the bottom 82″″″ of the piston 18″″″ reaches the side wall 160 of the third chamber 152″″″. Then, the contents of the third chamber 152″″″ is pressed through essential opening in the central pointed tip 92″″″ into the second chamber enclosed by the test container 14″″″. Venting of the second chamber enclosed by the test container 14″″″ can occur via venting tip 162.

For guiding a piston 18″″″, guiding ribs 164 are provided, that extend in the longitudinal direction of the lysis container 12′″″.

Lysis container 12″″″ may provide a guiding extension 166 for guiding the piston 18″″″. A hole 168 in the extension 166 allows inserting a tip of a swab 170. When the piston 18″″″ is pushed into the lysis container 12″″″ after while a swab 170 extends through the hole 168 in the wall of the extension 166, the swab is cut in parts and the part comprising the genetic material to be lysed is kept in the lysis container 12″″″; see FIG. 13. Note that the embodiment of FIG. 13 corresponds to the embodiment of FIG. 12.

In alternative embodiments, a cross slide for cutting the swap may be provided, the cross slide would have a cutting edge and would be arranged to move in a direction that is perpendicular to the longitudinal direction of the lysis container. Cross slide guiding elements may be provided on the lysis container for guiding the cross slide. Preferably, the cross slide is arranged close to an end of the lysis container that faces away from the test container.

Alternative embodiments of integral cutting means for cutting a swab can be provided with the other embodiments disclosed in this description to thus provide further preferred embodiments.

In yet another embodiment (not shown in the figures), the lysis container is one part but consists of a lower hollow and an upper plain cylinder. The test container plugs into the hollow part of the lysis container from the bottom. The lysis container covers the test container on the edges. When the lysis container is full, the hollow cylinder is filled and the plain cylinder is filled with 50 μl to 70 μl. Now the surface where the two containers meet needs to be opened and pushed to the edges, covering the liquids in the hollow cylinder but allowing the amount of 50 μl to 70 μl in the plain cylinder to mix with the pellets. The surface of the inner circle and the outer ring (of the hollow cylinder) must be equivalent The required amount (50 μl to 70 μl) is roughly the volume of a water-droplet.

In yet another embodiment (not shown in the figures), the lysis container is connected to the test container with an M-shaped tube. The “M” gets filled and when you turn it back around, liquid remains in the middle curvage. That amount can be calculated and used for further steps.

An embodiment similar to the embodiment in FIG. 6 is shown in FIGS. 14 to 17.

Lysis chamber 120 and test chamber 140 are hold in a housing 980 that is comprised of two parts, a lower housing part 98.1° and an upper housing part 98.2°. The lower housing part 98.1° has a flared side wall 180 that widens towards the bottom of housing 98°. The flared side wall 180 is configured to fit to a truncated cone shaped protrusion 182 of the detection device 160 (see FIGS. 18 and 19).

The top of housing 980 is closed by a cap 1100 that can swivel into an open position (see FIG. 14b) for selectively opening a central opening 184 for inserting a swab into the lysis chamber 130 of lysis container 12°.

In the upper part of housing 980 the lysis container 120 is held by means of a releasable snap fit connection achieved by a outwardly extending circumferential collar 186 around an upper section of the lysis container outer wall and two inwardly extending circumferential rips 188 on the inner wall of an upper section of housing 98°. The outwardly extending circumferential collar 186 of the lysis container 120 is held in a grove between the two inwardly extending circumferential rips 188 on the inner wall of housing 98°. The snap fit connection between the lysis chamber 120 and the housing 980 can be released by a force acting on the lysis chamber 120 in an axial direction that exceeds a predetermined threshold as provided by the design of the snap fit connection as provided inter alia by the matching shapes and the elastic properties of the materials and shapes.

In the bottom 840 of the lysis container 120 a hollow needle 200 is arranged. The needle 200 preferably is made from stainless steel.

Similar to the embodiment of FIG. 6, the lower part of the lysis container 120 including the lysis container bottom 840 is arranged to extend into a lysis container receptacle 1000 that is formed by a test container extension 1020 extending upwardly from an upper part of the test container 14. The outer diameter of the lower part of the lysis container 120 corresponds to the inner diameter of the lysis container receptacle 100°.

Similar to the embodiment of FIG. 6, a rotation of the housing 98 relative to the test container 14 causes a relative axial movement of the test container 14. This is achieved by at least one helical grove 202 that is formed in the inner side of the wall of the housing 98. In the illustrated embodiment, three helical groves 202 are provided. A radial protrusion 204 on the test container extension 1020 radially extends into the grove 202. Thus, the grove 202 acts as a helical slotted link guide for the radial protrusion 204 on the test container extension 102°. The helical grove 202 causes an axial movement of the test container 14 when the housing 980 is rotated while the test container 140 does not rotate. Accordingly, the helical grove 202 and the radial protrusion 204 are an alternative to the threading 104 of the embodiment of FIG. 6.

In order to prevent a rotation of the test container 140 when the housing 980 is rotated, a short longitudinal rip 206 extending in the radial direction from a wall of the test container 140 is provided. The rip 206 engages with a notch 208 of the receptacle 220 of the detection device 16° when the test container 140 of fluid tight assembly 340 is inserted on the receptacle 220 of the detection device 16°.

Rotating of the housing 980 relative to the test container 140 causes an axial movement of the test container 140 towards the lysis container 12°. This in turn causes the needle 200 to pierce the elastomeric septum 280 (i.e. the lid) of the test container 14°. Once the needle 200 at the bottom 840 of lysis container 120 pierces the elastomeric septum 28° (separating wall acting as a lid for the test container that can be pierced by a needle) that initially closed the second chamber in the test container 14°, fluid can be transferred from the lysis container 120 into the test container 14°.

The axial movement of the test container 140 towards the lysis container 120 further causes a compression of the lysis container 120 and thus a transfer of fluid from the lysis chamber 130 into the test chamber 15°. Further rotating of housing 980 causes further compression of the lysis container 120 until the axial forces on the lysis container 120 causing the compression of the lysis container 120 exceed the force needed to release the lysis container 120 from being held by the outwardly extending circumferential collar 186 of the lysis container 120 in the grove between the two inwardly extending circumferential rips 188 on the inner wall of housing 98°. Once released, the lysis container 120 can freely move upwards (i.e. in the direction towards the central opening 184 at the top of housing 98°) and the lysis container 120 is not further compressed. Thus, further transfer of fluid from the lysis chamber 13° into the test chamber 15° is stopped. Compression of the lysis container 12° is thus limited by the force needed for pushing the outwardly extending circumferential collar 186 of the lysis container 120 out of the grove between the two inwardly extending circumferential rips 188 on the inner wall of housing 98°.

For using the fluid tight assembly 340 in combination with the detection device 16°, first a swap with a sample to be tested is inserted in the lysis camber 130 via the central opening 184. Next, cap 1100 is closed and lysis can occur in the lysis chamber 13°. Once lysis has occurred, the fluid tight assembly 340 can be engaged with the detection device 160 by inserting the test container 140 into the receptacle 220 of the detection device 16°. This is facilitated by the flared side wall 180 of the lower section of housing 98°.

To further facilitate engaging the fluid tight assembly 340 with the detection device 160 in the right orientation that allows the short longitudinal rip 206 of the test container 140 to engage with the notch 208 of the receptacle 220 of the detection device 16°, a palpable raised rip 210 is provided on the outer surface of the housing 98°. The palpable raised rip 210 runs along a longitudinal direction of the housing 98°.

A palpable protrusion 212 on the detection device 160 next to the truncated cone shaped protrusion 182 of the detection device 160 is a further palpable feature that helps orienting the palpable raised rip 210 and thus the fluid tight assembly 340 correctly.

Once engaged with the detection device 16°, the housing 980 of the fluid tight assembly 340 can be rotated to cause the relative axial movement of the test container 140 relative to the lysis container 120 and the housing 98°. Since immediately after engaging the fluid tight assembly 340 with the testing device 160 the test container 140 already is fully inserted into the receptacle 220 and thus the detection chamber 42° of the detection device 16°, the housing 980 is axially spaced from the detection device 16°. By rotating the housing 980 relative to the detection device 160 and thus the test container 14°, the axial distance between the housing 980 and the detection device 160 is minimized until the housing 980 touches the detection device 160 and the needle 200 has pierced the septum 280 of the test container 14°. As seen from outside, while rotating the housing 980 clockwise, the housing 980 moves downwardly thus approaching the detection device 16°.

Once the housing 980 is rotated in its final position, a further effect of the truncated cone shaped protrusion 182 of the detection device 160 in combination with the flared side wall 180 of the housing 980 is an improved protection from straylight entering the detection chamber 420 of the detection device 16°.

The detection chamber 420 of the fluorescence detection device 160 is configured to receive the test container 14°. The receptacle 220 is part of the detection chamber 42°. The light source 440 comprises two light emitting diodes that are arranged to illuminate the contents of the test chamber 150 in the detection chamber 420 with a light that can cause luminescence in a sample to be tested during and after the sample has undergone recombinase polymerase amplification. The optical sensor 460 is arranged and configured to detect luminescence in the detection chamber 420 in case luminescence occurs.

For controlling the light emitting diodes 440 and reading out and processing the signal provided by the light sensor 46°, a controller 500 is provided that is operatively connected to a wireless data interface 520 comprising an NFC antenna.

Energy is supplied by a recharge battery 480 that can be charged via an USB-C port 214.

Yet another embodiment of a fluid tight assembly is illustrated in FIGS. 20a, 20b and 20c, a third container 220 is provided that serves for collecting a liquid sample instead of a sample collected by means of a swab.

The third container can be a separate sample container 220 that is used for taking a sample for instance by way of gargling or mouthwash. The sample container can be connected to a lysis container 12^{vii} and a test container 14^{vii}. The lysis container 12^{vii} and the test container 14^{vii} can be configured as disclosed herein above. A combination of a lysis container and a test container is called reaction containers hereinafter. The reaction containers, i.e. the test container 14^{vii} and the lysis container 12^{vii} can form a single unitary unit while the sample container can be a separate component that can be attached to the reaction containers.

The sample container 220 comprises a sample chamber 222 that can be closed by a sample container lid 224.

The sample chamber can be initially empty or it may contain a liquid such as pure water, a solution containing H₂O₂ or the like that can be used for gargling or mouth washing.

The sample container 220 is used for sampling while lysing and the test reaction, for instance the amplification, is performed in the reaction containers, i.e. the lysis container 12^{vii} and the test container 14^{vii}, respectively.

The correct dosing of the sample liquid is done through and during the connection of the two separate parts.

The containers can be connected, for example, by plugging them together or by a rotary movement, e.g. screwing, or a combination of both, for instance by way of a bayonet connection. Preferably, the containers are designed so that the movement for connecting the containers (plugging, screwing etc.) trigger a transfer of a defined amount of sample liquid to the reaction containers, thus triggering the detection reaction.

Preferably, in a first step, sample liquid is transferred from the sample container 220 into the lysis chamber in the lysis container 14^{vii}.

In second step, after lysing has occurred, liquid is further transferred to the test chamber of test container 14^{vii}. The amount of liquid transferred into the test chamber preferably is less than 100 μl, more preferred less than 50 μl. The amount depends and is adapted to the respective test.

In one embodiment, the sample container contains the liquid for sampling. A test person can take the liquid up for rinsing the mouth. Thereafter, the liquid comprising the sample is put back into the sample container. Then, the sample container is closed and can be connected with the reaction containers 12^{vii} and 14^{vii}.

Typically, the sample liquid initially contained in the sample chamber is such that it can be spat into the mouth of test persons without danger and that it can collect the sample obtained by mouthwash.

According to a first embodiment, the sample liquid contained in the sample container can be a liquid that can lyse the viruses or bacteria so that a separate lysis step is no longer necessary. This could typically be realized by an oxidation reaction, e.g. by a 1-3% H₂O₂ solution. In such embodiment, no separate lysing chamber is needed.

In yet another embodiment, no liquid is kept in the sample container for sampling, the test person rinses his mouth with cold tap water and spits this rinsing liquid into the sample container. The sample container also contains another liquid for the lysis of the sample.

The two liquids are only brought together after the sampling has taken place, i.e. the test person has spit the rinsing liquid into the sample container. In this embodiment, separate compartments are provided in the sample container to separate the lysing liquid from the sample liquid before mixing. By means of a mechanical, thermal or chemical process the two liquids can be treated in such a way that the liquids mix homogeneously after collecting the sample has occurred.

In both cases, the sample container and the reaction containers are connected to each other by plugging or twisting, e.g. by a bayonet fastener 226 as shown in FIG. 20b). In one embodiment, the arrangement of the lysis chamber and the sample container can be designed such that the sample container and lysis chamber are arranged on top of each other, as shown in FIG. 14. In alternative embodiments the containers are designed to be arranged side by side. In yet another embodiment, already lysed sample can be added to the sample container, thus avoiding a separated lysing step.

When connecting the containers, a mechanism is actuated which allows a transfer of a defined amount of liquid (of the sample) into the reaction containers. The reaction can then take place. Fluid transfer means 228—as seen in the top view depicted in FIG. 20b)—allow the transfer of fluid.

1. A set of at least two distinct containers that can be combined to form a single, fluid tight assembly, wherein a first container encloses at least a first chamber containing a first set of chemicals and/or agents and wherein a second container comprises at least a second chamber containing a second set of chemicals and/or agents that are at least in part distinct from the chemicals and/or agents of the first set of chemicals and/or agents, and wherein the first container and/or the second container comprises a septum, that initially is fluid tight and that can be altered to form a passageway between the first container and the second container when the first container and the second container are combined to form a single, fluid tight assembly, in order to allow a transfer of fluid from the first container to the second container.

2. The set of containers according to claim 1, wherein the first container contains more fluid than shall be transferred to the second container and wherein either the first container or the second container or the combination of the first and the second container comprises flow control means that are configured to allow a limited transfer of fluid from the first container into the second container.

3. The set of containers according to claim 2, wherein the first container is provided with manually actuated volume reducing means that are configured to cause a defined reduction of the first container fluid volume when actuated to thus squeeze a defined amount of fluid from the first container into the second container.

4. The set of containers according to claim 2, wherein the first container encloses two chambers, a first and a third chamber, that initially are fluidly connected to one another and that can be separated from one another by a movable member, wherein the third chamber has a volume that corresponds to the amount of fluid to be transferred from the first container into the second container.

5. The set of containers according to claim 1, wherein the first chamber has a volume of at least 100 μl and wherein volume to be transferred is smaller than 80 μl.

6. The set of containers according to claim 1, wherein the containers can be connected by means of a fluid tight snap fit connection.

7. The set of containers according to claim 1, wherein the second container has transparent walls.

8. The set of containers comprising at least one test container (14^{vii}) and a sample container that is configured to for collecting a fluid sample.

9. The set of containers according to claim 8, comprising a sample container, a lysis container (12^{vii}) and a test container (14^{vii}).

10. The set of containers according to claim 9, wherein the sample container comprises a sample chamber and a compartment for a lysing fluid, said compartment initially being separated from the sample chamber.

11. The set of containers according to claim 1, wherein the first container and the second container are arranged in a housing that comprises means for altering the distance between the first container and the second container so as to selectively provide a first relative distance between the first container and the second container wherein the first chamber and the second chamber enclosed by the respective first container and second container are fluidly separated and a second relatively closer distance between the first container and the second container wherein the first chamber and the second chamber are in fluid communication.

12. The set of containers according to claim 11, wherein the means for altering the distance between the first container and the second container comprise a helical member, in particular a helical grove that is configured to translate a rotation into an axial movement of one container with respect to the other container.

13. The set of containers according to claim 11, wherein the first container is held in the housing by a releasable snap fit connection that is released if a force exerted by the second container on the first container exceeds a predetermined threshold.

14. A system for detecting target DNA in a biological sample, said system comprising a first container, a second container and a detection device, wherein:
the first container contains a lysing fluid,
the second container contains a mixture that comprises a recombinase, a single-stranded DNA-binding protein, strand-displacing polymerase and exonuclease, and
the detection device comprises:
a detection chamber configure to receive a test container or the contents of the test container,
a light source configured to excite luminescence and in particular fluorescence,
an optical sensor configured to capture luminescence and on particular fluorescence,
energy supply means,
a wireless data interface, and
a controller.

15. The system according to claim 14, wherein the detection device further comprises a receptacle that is formed to receive the second container.

16. The system according to claim 12, wherein the detection device and the second container comprise mutually engaging features that prevent a relative rotation between the detection device and the second container.
